(19) United States
(12) Patent Application Publication
(10) Pub. No.: US 2024/0315763 A1
(43) Pub. Date: Sep. 26, 2024

(54) HANDPIECE FOR A SURGICAL INSTRUMENT

(71) Applicant: GYRUS MEDICAL LIMITED, Cardiff (GB)

(72) Inventors: Christopher Charles BENN, Cardiff (GB); David MORRIS, Cardiff (GB)

(73) Assignee: GYRUS MEDICAL LIMITED, Cardiff (GB)

(21) Appl. No.: 18/603,771

(22) Filed: Mar. 13, 2024

Related U.S. Application Data
(60) Provisional application No. 63/454,172, filed on Mar. 23, 2023.

(51) Int. Cl. A61B 18/14; A61B 18/00; A61B 18/12

(52) U.S. Cl. CPC A61B 18/148; A61B 18/1206; A61B 2018/00011; A61B 2018/00101; A61B 2018/00577; A61B 2018/00589; A61B 2018/0091

(57) ABSTRACT

The present disclosure seeks to provide a handpiece configuration for a surgical instrument such as an electrosurgical instrument to reduce the risk of a burn to the operator handling the handpiece. In particular, the present disclosure firstly provides an inner moulding in the handpiece for housing a motor and a suction path of the electrosurgical instrument. The inner moulding comprises a thermal conduction path between the motor and the suction path for the purpose of transferring heat generated from the motor to the exiting irrigation fluid in the suction path. The handpiece is further configured to comprise a thermally insulating outer casing surrounding the inner moulding. This configuration of the handpiece enables heat transfer from the motor to the irrigation fluid in the suction path while keeping the external surface of the handpiece cool.

### TECHNICAL FIELD

The present invention relates to surgical instruments, such as for example electrosurgical instruments. More specifically, the present invention relates to a handpiece for an electrosurgical instrument.

### BACKGROUND

Surgical instruments, including radio frequency (RF) electrosurgical instruments, have become widely used in surgical procedures where access to the surgical site is restricted to a narrow passage, for example, in minimally invasive “keyhole” surgeries.

RF electrosurgical instruments typically include an active electrode which forms a distal RF tip of the instrument, and a return electrode. The distal RF tip provides tissue ablation and/or coagulation effects at a surgical site when a RF power signal is delivered to the electrodes. Some RF instruments also implement a mechanical shaver, with the main mechanical shaving componentry located on the opposite side of the distal tip.

An electrosurgical instrument typically comprises a motor disposed within the handpiece for controlling the operation of the instrument. A motor within a handpiece of the instrument helps to control the surgical operations of the instrument, for example, shaving operation of the mechanical shaver. For example, the motor may be used to drive the mechanical shaver to remove tissue and bone in a fluid/wet-field within the body. The electrosurgical instrument may also comprise a suction path for extracting irrigation fluid, for example, saline from the wet-field environment during the operation of the instrument.

In a conventional electrosurgical instrument, heat generated from the motor during operation of the instrument is dissipated to the waste irrigation fluid being extracted via the suction path in the handpiece. The heat transfer from the motor to the waste irrigation fluid is achieved using a metallic handpiece. A drawback of using a metallic handpiece is that, if the flow of the waste irrigation fluid decreases, the heat transferred from the motor remains within the handpiece causing the temperature of the handpiece to increase. This in turn causes a risk of a burn to an operator using the metallic handpiece.

U.S. Pat. No. 11,426,231 B2 discloses a handpiece having a channel for outflow of saline wherein a thin wall sleeve is located in the handpiece so that it surrounds at least a portion of the said channel. The thin wall sleeve is surrounded by an air gap between an exterior surface of the thin wall sleeve and an inner surface of the body in order to limit heat transfer from the heated or other fluid outflow through the channel.

However, there exists a need for an improved handpiece configuration for reduced risk of burn to an operator using the handpiece.

### SUMMARY OF THE INVENTION

The present disclosure seeks to provide a handpiece configuration for a surgical instrument, and in particular though not exclusively an electrosurgical instrument, to reduce the risk of a burn to the operator handling the instrument. In particular, the present disclosure firstly provides an inner moulding in the handpiece for housing a motor and a suction path of the surgical instrument. An outer casing formed of a thermally insulative material surrounds the inner moulding. The present disclosure also provides a thermal conduction path between the motor and the suction path for the purpose of transferring heat generated from the motor to the exiting irrigation fluid in the suction path. The proposed configuration of the handpiece enables heat transfer from the motor to the irrigation fluid in the suction path while keeping the external surface of the handpiece cool. An additional advantage of an outer casing made of a thermally insulative material is that it is lighter in weight compared to the metallic outer casing in a conventional handpiece. The term electrosurgical instrument as used in this description refers to an RF surgical instrument which may also include a combined shaver.

According to a first aspect of this invention, a handpiece for a surgical instrument is provided, the handpiece comprising: an inner moulding for housing a motor and at least one channel configured to be disposed adjacent to the motor for draining irrigation fluid during operation of the said instrument; an outer casing surrounding the inner moulding, the outer casing formed of a first thermally insulative material, wherein the inner moulding further comprises a first region configured to enable thermal coupling between the motor and the at least one channel, the first region being formed of a thermally conductive material.

According to a second aspect of this invention, an electrosurgical instrument is provided, wherein the electrosurgical instrument comprises: a handpiece according to the first aspect of this invention, an elongate shaft extending from a distal end of the hand-piece; and an end effector positioned at a distal end of the elongate shaft.

According to a third aspect of this invention, an electrosurgical system is provided, wherein the system comprises: an RF electrosurgical generator; and an electrosurgical instrument according to the second aspect of this invention.

According to a fourth aspect of this invention, a motor for controlling an end effector of a surgical instrument is provided, the motor comprising: a thermally conductive region disposed on an outer surface of the motor, the thermally conductive region being configured to receive at least one channel of the electrosurgical instrument and to enable thermal coupling between the motor and the at least one channel.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 shows an electrosurgical system including an electrosurgical instrument, according to an embodiment of the present disclosure.

FIG. 2A illustrates an example of a handpiece for an electrosurgical instrument according to an embodiment of the present disclosure;

FIG. 2B illustrates a cross-section through the line A-A′ of the handpiece in FIG. 2A.

### DETAILED DESCRIPTION OF THE EMBODIMENTS

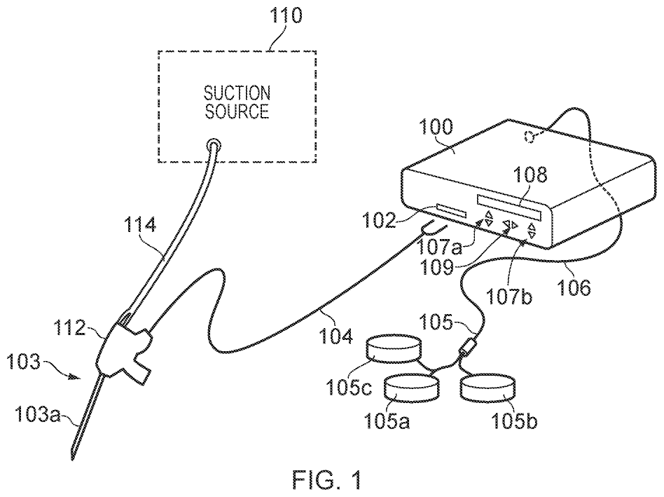

FIG. 1 shows an electrosurgical apparatus including an electrosurgical generator 100 having an output socket 102 that provides a radio frequency (RF) output (e.g. a RF power signal), via a connection cord 104, to an electrosurgical instrument 103 having an end effector 103a that may be configured to provide a mechanical shaving function, as well as a electrosurgical cutting and coagulation functions. The instrument 103 has a suction tube 114 which is connected to a suction source 110. Activation of the generator 100 may be performed from the instrument 103 via a hand switch (not shown) on the handpiece 112 of the instrument 103, or by means of a footswitch unit 105 connected separately to the generator 100 by a footswitch connection cord 106. In the illustrated embodiment, the footswitch unit 105 has three footswitches 105a, 105b and 105c for selecting a mechanical shaving mode, a coagulation mode, or a cutting or vaporisation (ablation) mode of the generator 100 respectively. The generator front panel has touch screen buttons 107a and 107b for respectively setting ablation (cutting) or coagulation power levels, which are indicated in a display 108. Touch screen buttons 109 are provided as an alternative means for selection between different modes.

As described above, the electrosurgical instrument 103 may be a dual sided (or an opposite sided) RF shaver device. In this respect, the main RF componentry and the manual shaving/cutting componentry of the instrument 103 can be provided on opposite sides of a distal end portion of the instrument 103. However, it will also be appreciated that the electrosurgical instrument 103 may be an RF device providing only electrosurgical cutting and coagulation functions.

The electrosurgical instrument 103 and/or end effector 103a used in conjunction with the handpiece disclosed herein can be designed to be disposed of after a single use, or they can be designed to be used multiple times. In either case, however, the electrosurgical instrument and/or end effector can be reconditioned for reuse after at least one use.

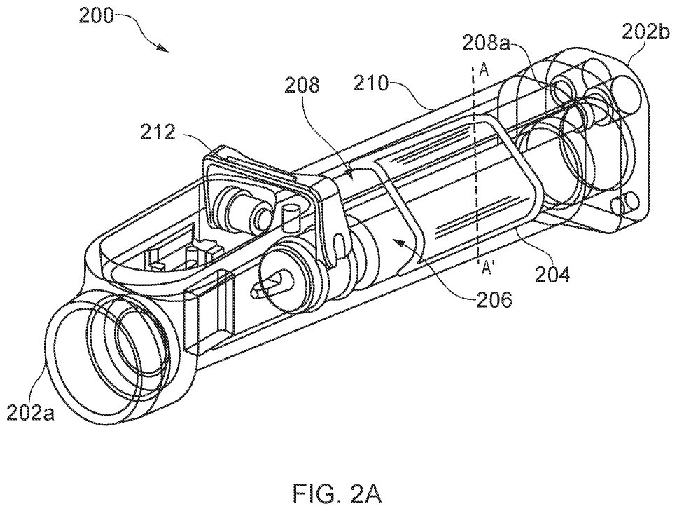

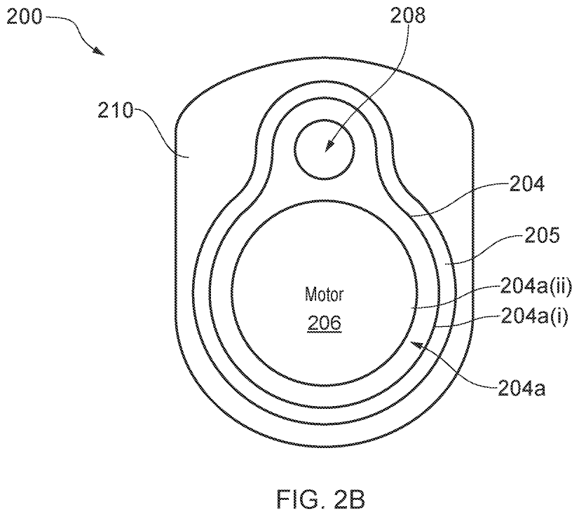

FIG. 2A shows a perspective view of a handpiece 200 according to an embodiment of this disclosure. The handpiece 112 in FIG. 1 is configured according to FIG. 2A. The handpiece 200 has a first end 202a for coupling to an end effector, for example the end effector 103a in FIG. 1. The handpiece 200 has an inner moulding 204 configured to house a motor 206 and a suction path 208 disposed adjacent to the motor 206 to enable thermal coupling between the motor 206 and suction path 208. The handpiece 200 also comprises a thermally insulative outer casing 210 surrounding the inner moulding 204. In some embodiments, the handpiece 200 may be configured to have a plurality of suction paths disposed adjacent to the motor 206.

The motor 206 is configured to control the operation of the end effector 103a (see FIG. 1), for example, a mechanical shaving operation of the end effector 103a. The suction path 208 is configured to be coupled to a suction source 110 to provide an exit path for the waste irrigation fluid when the electrosurgical instrument is used in a fluid-filled field. The skilled person in the art will understand that saline is a commonly used irrigation fluid in electrosurgical instruments.

The suction path or channel 208 can also be referred to as a fluid or saline drainage path. In FIG. 2A, the distal end 208a of the suction path 208, located at a second end 202b of the handpiece 200, is configured for coupling to the suction source 110. The handpiece 200 may also comprise a handle 212.

In conventional electrosurgical instruments, the handpiece of the electrosurgical instrument has a metallic outer casing. A drawback of using a metallic handpiece is that, if the flow of the waste irrigation fluid through the suction path decreases, the heat transferred from the motor remains within the handpiece causing the temperature of the handpiece to increase. This in turn causes a risk of a burn to an operator using the metallic handpiece.

The inventors of the present invention have realised that the handpiece 200 can be configured to provide a thermally insulative outer casing to prevent thermal coupling or heat transfer from the motor 206 to the outer casing 210 of the handpiece 200. The inventors have proposed to further configure the inner moulding of the handpiece 200, as will be described below, to enable the heat generated by the motor to be dissipated to the waste irrigation fluid in the suction path 208.

The proposed configuration of the handpiece 200 will now be described in detail with reference to FIG. 2B.

FIG. 2B shows a cross-sectional view of the handpiece 200 through line A-A′ in FIG. 2A with the inner moulding 204 and the thermally insulative outer casing 210 surrounding the inner moulding 204. The provision of a thermally insulative outer casing 210 prevents thermal coupling between the inner moulding 204 and the outer casing 210. As seen in FIG. 2B, the inner moulding 204 provides a housing for the motor 206 and the suction path 208 disposed adjacent to the motor 206 for draining irrigation fluid during operation of the electrosurgical instrument.

The inventors have proposed to configure the inner moulding 204 such that the inner moulding 204 comprises a first region 204a. The first region 204a is formed of a thermally conductive material and is configured to enable thermal coupling or heat transfer between the motor 206 and the suction path 208 during the operation of the electrosurgical instrument. This enables the waste irrigation fluid, for example saline, exiting out of the suction path 208 to be heated thereby providing a mechanism for dissipating the heat generated by the motor 206 during the operation of the electrosurgical instrument. The thermally conductive material of the first region 204a may be formed of a metal selected from the group comprising stainless steel, copper, aluminium, aluminium alloys, titanium or a combination thereof.

In the embodiment of FIG. 2B, the handpiece 200 comprises an optional second region 205 disposed between the inner moulding 204 and the thermally insulative outer casing 210. In embodiments where the second region 205 is provided, for example, as shown in FIG. 2B, the second region 205 surrounds the inner moulding 204 and is formed of a thermally insulative material. For example, as seen in FIG. 2B, the second region 205 is formed on the outer surface of the inner moulding 204. As the second region 205 is thermally insulative, it prevents thermal coupling between the inner moulding 204 and the outer casing 210—in particular, the second region 205 prevents the transfer of heat to the outer casing 110 from the motor 206 and the heated irrigation fluid exiting via the suction path 208, during operation of the electrosurgical instrument. The second region 205 can be an air gap or an aerogel layer.

In other embodiments, the handpiece can be configured to omit the second region 205 such that the thermally insulative outer casing 210 surrounds the inner moulding 204 and is in physical contact with the outer surface of the inner moulding 204 having the first region 204a.

As a result of the above proposed configuration of the handpiece 200, in particular the provision of a thermally insulative outer casing 210 and an inner moulding 204 having a first thermally conductive region 204a, heat generated by the motor 206, during operation of the electrosurgical instrument, is dissipated to the waste irrigation fluid exiting the suction path 208 but is not transferred to the thermally insulative outer casing 210. In this way, the proposed configuration of the handpiece 200 avoids the risk of a burn to an operator using the handpiece. The provision of an optional second thermally insulative region 205 between the outer casing 210 and the inner moulding 204 further helps to prevent thermal coupling between the inner moulding 204 and the outer casing 210. The outer casing 210 is formed of a thermally insulative material such as polyether ether ketone (PEEK) or ceramic, e.g. alumina—this provides an additional advantage of the outer casing being lighter in weight compared to the metallic outer casing in a conventional handpiece.

In embodiments where the second region 205 is provided, the thermally insulative material used for the outer casing 210 is different from the thermally insulative material used for the second region 205.

The thickness of the first region 204a, defined by a distance between the inner wall 204a(ii) and outer wall 204a(i) of the first region 204a in FIG. 2B, does not necessarily have to be uniform throughout. For example, in some embodiments, a thickness of the first region 204a may be configured to be optimised for thermal coupling in the region between the motor 206 and the suction path 208 when compared to a thickness of the remainder of the first region 204a.

In some embodiments, the first region 204a may be configured to extend only along a length of the heat-generating portion, for e.g. coils and gearbox, of the motor 206. In some embodiments, where the second region 205 is also provided, the second region 205 and the first region 204a may be configured to extend only along a length of the heat-generating portion of the motor 206.

While FIG. 2B shows the first region 204a completely surrounding the motor 206 and the suction path 208, other variations are also possible for the circumferential coverage of the first region 204a while still enabling heat transfer between the motor 206 and the suction path 208.

For example, in some embodiments, the first region 204a, while being configured to enable heat transfer between the motor 206 and the suction path 208, may only be partially disposed around the motor 206 while completely surrounding the suction path 208. In some embodiments, the first region 204a, while being configured to enable heat transfer between the motor 206 and the suction path 208, may partially surround the motor 206 and partially surround the suction path 208.

In some other embodiments, the first region 204a, while being configured to enable heat transfer between the motor 206 and the suction path 208, may partially surround the suction path 208 and completely surround the motor 206.

For example, the first region 204a, while being configured to enable heat transfer between the motor 206 and the suction path 208, may extend around the circumferential area of the motor 206 over an angle in the range between 100° to 360°, preferably between 120° to 300° and more preferably between 180° to 270°. Similarly, the first region 204a may extend around the circumferential area of the suction path 208 over an angle in the range between 100° to 360°, preferably between 120° to 300° and more preferably between 180° to 270°.

While the handpiece 200 may be formed with the first region 204a and suction path 208 integrated into the handpiece 200, in another embodiment, the first region 204a may be integrated with the motor 206 itself. That is, a motor 206 may be configured to comprise the first region 204a on the outer casing of the motor. In this case, the first region 204a is further configured to receive the suction path 208 such that when the handpiece 200 is assembled with the motor 206, the resulting configuration is the same as in FIG. 2B or the different embodiments of the handpiece as described above.

Various further modifications to the above-described embodiments, whether by way of addition, deletion, or substitution, will be apparent to the skilled person to provide additional embodiments, any and all of which are intended to be encompassed by the appended claims.

1. A handpiece for a surgical instrument comprising:
an inner moulding for housing a motor and at least one channel configured to be disposed adjacent to the motor for draining irrigation fluid during operation of the said instrument;
an outer casing surrounding the inner moulding, the outer casing formed of a first thermally insulative material,
wherein the inner moulding further comprises a first region configured to enable thermal coupling between the motor and the at least one channel, the first region being formed of a thermally conductive material.

2. A handpiece according to claim 1, wherein the handpiece comprises a second region disposed between the inner moulding and the outer casing, the second region being formed of a second thermally insulative material and configured to surround the inner moulding to prevent thermal coupling between the inner moulding and the outer casing.

3. A handpiece according to claim 1, wherein the first region is further configured to partially surround the at least one channel and partially surround the motor.

4. A handpiece according to claim 1, wherein the first region is further configured to partially surround the at least one channel and completely surround the motor.

5. A handpiece according to claim 1, wherein the first region is further configured to completely surround the at least one channel and partially surround the motor.

6. A handpiece according to claim 3, wherein the first region extends over an angle between 120° to 300° around the circumferential area of the at least one channel.

7. A handpiece according to claim 6, wherein the first region extends over an angle between 180° to 270° degrees around the circumferential area of the at least one channel.

8. A handpiece according to claim 3, wherein the first region extends over an angle between 120° to 300° around the circumferential area of the motor.

9. A handpiece according to claim 8 wherein the first region extends over an angle between 180° to 270° degrees around the circumferential area of the motor.

10. A handpiece according to claim 1, wherein the first region has a non-uniform thickness.

11. A handpiece according to claim 1, wherein the first region is configured to extend along a length of a heat-generating portion of the motor.

12. A handpiece according to claim 1, wherein the first thermally insulative material comprises polyether ether ketone (PEEK) or ceramic.

13. A handpiece according to claim 2, wherein the second thermally insulative material comprises air or an aerogel layer.

14. A handpiece according to claim 1, wherein the thermally conductive material is selected from a group comprising stainless steel, copper, aluminium, aluminium alloys, titanium or a combination thereof.

15. A handpiece according to claim 1, wherein the handpiece comprises a plurality of channels disposed adjacent to the motor.

16. An electrosurgical instrument comprising:
a handpiece according to claim 1,
an elongate shaft extending from a distal end of the hand-piece; and
an end effector positioned at a distal end of the elongate shaft.

17. An electrosurgical system, comprising:
an RF electrosurgical generator; and
an electrosurgical instrument according to claim 16.

18. A motor for controlling an end effector of a surgical instrument, the motor comprising:
a thermally conductive region disposed on an outer surface of the motor, the thermally conductive region being configured to receive at least one channel of the surgical instrument and to enable thermal coupling between the motor and the at least one channel.

19. A motor according to claim 18, wherein the thermally conductive region is further configured to:
a. partially surround the at least one channel and partially surround the motor; or
b. partially surround the at least one channel and completely surround the motor.

20. A motor according to claim 18, wherein the thermally conductive region is further configured to completely surround the at least one channel and partially surround the motor.
